(19) United States
(12) Patent Application Publication
(10) Pub. No.: US 2008/0152626 A1
(43) Pub. Date: Jun. 26, 2008

(54) Method for Activating Cd8 T Cells

(75) Inventors: Yong-Keun Park, Seoul (KR); Won-Suck Yoon, Seoul (KR)

Correspondence Address: THE RAFFERTY PATENT LAW FIRM, 5641 BURKE CENTRE PKWY, SUITE 100, BURKE, VA, 22015-2259, US

(73) Assignee: Korea University Industry and Academy Cooperation Foundation, Seoul (KR)

(21) Appl. No.: 11/912,150

(22) PCT Filed: Jan. 20, 2006
(86) PCT No.: PCT/KR06/00228

§ 371 Date: Oct. 21, 2007

(30) Foreign Application Priority Data
Apr. 21, 2005 (KR) 10-2005-0033191

(51) Int. Cl. A61K 35/12; C12N 5/02; A61P 31/12

(52) U.S. Cl. 424/93.7; 435/377

(57) ABSTRACT

A method for activating a CD8 T cell includes separating CD8 T cells and cultivating them in vitro with a culture broth containing cytokines such as GM-CSF, IFN-γ, TNF-α, lectin, IL-2 and IL-4. Also provided is a therapeutic composition for preventing or treating infectious viral diseases, which includes the CD8 T cell activated by the above-noted method.

### CROSS-REFERENCES TO RELATED APPLICATIONS

This is a US National Stage of International Application PCT/KR2006/000228, filed with the Republic of Korea Receiving Office on Jan. 20, 2006. The benefit of priority is further claimed to Republic of Korea patent application 2005-0033191, filed Apr. 21, 2005.

### TECHNICAL FIELD

The present method relates to activating a CD8 T cell, and may include separating CD8 T cells, and cultivating them in vitro with a culture broth containing cytokines such as GM-CSF, IFN-γ, TNF-α, lectin, IL-2 and IL-4. Also provided is a therapeutic composition for preventing or treating infectious diseases of virus, which includes the CD8 T cell activated by the above-noted method.

### BACKGROUND

Influenza virus is a pathogenic and causative agent of severe bouts of the flu. The virus lives in somatic cells parasitically and, after the parasitism, disrupts the cells pathologically for its own proliferation.

Because the virus is parasitic on the somatic cell, drugs showing direct medical effect on the viral infection have not been developed. Rather, the viral infection has been treated using an agent relieving the symptom of chill, vomiting or fever which accompanies with the viral infection while relying on the medical effect of the host's natural immune system.

Recently, a zinc gel spray-type drug was developed which is directly administered to the nasal mucosa. However, the drug induces a medical effect by preventing the influenza virus from attaching to the cell surface rather than by directly treating the virus.

In the research field of drugs, immune cells, etc., are being investigated to develop a therapeutic agent. For this therapeutic method, the immune cells are collected from a particular subject and then activated before being administered to a patient directly. Presently, immune cells activated by APC cell and specific antibody, etc., have been chosen for therapeutic use.

### SUMMARY

In view of the above-mentioned and other problems, the present inventors have found that a CD8 T cell is activated synergistically by adding a culture broth containing GM-CSF, IFN-γ, TNF-α, lectin, IL-2 and IL-4 and further, the resulting CD8 T cell can treat infectious diseases of virus and prevent their re-infections effectively and completed the invention successfully.

An object of the present invention is to provide a method for in vitro activation of a CD8 T cell that includes separating CD8 T cells from a biological specimen of a subject, and cultivating them with a culture broth containing cytokines including GM-CSF, IFN-γ, TNF-α, lectin, IL-2 and IL-4.

Another object of the present invention is to provide a therapeutic composition for preventing or treating infectious viral diseases that includes the CD8 T cells activated by the above-noted method.

### BRIEF DESCRIPTION OF THE DRAWINGS

The above and other objects, features and advantages of the present invention will be more clearly understood from the following detailed description taken in conjunction with the accompanying drawings, in which:

FIG. 1 depicts the process for activating CD8 T cell after separating it from a subject schematically;

FIG. 2 depicts the comparison of cell efficacies on mice infected by Influenza virus;

FIG. 3 depicts the test of vaccine efficacy on Influenza infection in mice survived after administering the activated CD8 T cell; and

FIG. 4 depicts the antigen-antibody reaction against Influenza virus in sera collected from mice survived after administering the activated CD8 T cell.

### DETAILED DESCRIPTION

In one embodiment of the present invention, a method for in vitro activation of a CD8 T cell is provided that includes separating CD8 T cells from a biological specimen of a subject, and cultivating the CD8 T cells with a culture medium containing cytokines including GM-CSF, IFN-γ, TNF-α, lectin, IL-2 and IL-4. The T cell denotes a T cell population that participates in an immune reaction and expresses a specific marker on the cell surface. For example, in accordance with one embodiment, only a T cell having a CD8 marker is selected from among the T cell population. Preferably, the T cell having a CD8 marker may include a cytotoxic T cell (Tc cell) that directly removes virus-infected cells or cancer cells by lysing the cells, and/or a suppressor T cell (Ts cell).

In the description of the present invention, “subject” is a donor providing a mononuclear cell such as T cell and includes all organisms if having a vascular system and hematopoietic cell. Preferably, the subject can be a vertebrate selected among cow, horses, sheep, pigs, goat, camels, antelopes, dogs, mice and the like.

The T cell, that is to say the CD8 T cell can be isolated from various biological samples including mononuclear cell. Preferably, the T cell can be purified among blood, plasma, lymph node, spleen, thymus, bone marrow and the like.

The process for isolating the T cell is not limited. Preferably, the T cell can be isolated by depending upon cell density, affinity of antibody against cell surface epitope, cell size, degree of fluorescent emission. In detail, the T cell is isolated by conducting a density gradient centrifugation using albumin, dextran, Ficoll, metrizamid, Percoll and the like; (MACS) etc. using an antibody; a centrifugal elutriation etc. depending upon cell size; and a FACS using fluorescence.

In the present invention, the T cell is isolated by performing a magnetic activated cell sorter using an anti-CD8 T cell antibody in order to purify a CD8 T cell from a mononuclear cell exclusively.

The CD8 T cell isolated above is cultivated by a conventional method and in vitro activated. Preferably, the T cell is cultivated by using a culture medium containing nutrients essential for cell growth and survival. Preferably, the culture medium is comprised of various sources of carbon, nitrogen and trace elements; more preferably, a culture media containing serum; and most preferably, commercial media such as DMEM and RPMI.

In the description of the present invention, “activation of T cell” is to stimulate a T cell to participate in an immune reaction. The T cell can be activated by various signals. Considering the objects of the present invention, the activation of T cell denotes an activation of CD8 T cell.

The present inventors have confirmed that a CD8 T cell is activated synergistically by adding a culture mixture containing GM-CSF, IFN-γ, TNF-α, lectin, IL-2 and IL-4. In detail, experimental mice are observed to have 60% of survival rate, when they are injected with the CD8 T cell activated by cytokines according to the above-mentioned process, in spite of 100% of death rate by the infection of Influenza virus A. In contrast, experimental mice injected with the CD4 T cell do not have influence on the death rate even though delaying the time to death. As a consequence, it is noted that the CD8 T cell is very effective to treat infectious diseases caused by virus pertaining to Orthomyxoviridae family.

In addition, the experimental mice survived by injecting the CD8 T cell are observed to prevent re-infections of Influenza virus. As a consequence, it is noted that the CD8 T cell has an outstanding efficacy as a vaccine.

In the method for in vitro activating a CD8 T cell, the concentration of cytokines included in the culture broth are adjusted in the ranges of 0.05 to 0.2 μg/ml of GM-CSF, 0.5 to 2 μg/ml of IFN-γ, 0.05 to 0.2 μg/ml of TNF-α, 40 to 60 μg/ml of lectin, 0.05 to 0.2 μg/ml of IL-2 and 0.05 to 0.2 μg/ml of IL-4.

The cytokine is a wild type of GM-CSF, IFN-γ, TNF-α, lectin, IL-2 or IL-4 derived from various subjects, but can be a fragment or variant of GM-CSF, IFN-γ, TNF-α, lectin or IL-4, if maintaining a biological activity of the wild type.

The culture broth containing the cytokines can include additional ingredients for activating CD8 T cell. Preferably, the additional ingredient can be IL-12, FLT3-ligand, Lipopolysaccharide (LPS) and the like.

Within an effective dose, the process adding the cytokine mixture into a culture broth, the timing, and the number of addition are not particularly limited in order to activate the CD8 T cell. The cytokine mixture can be added while the CD8 T cell is cultivated and otherwise, several times in some interval after cultivated. In addition, within an effective dose, the cytokine mixture can be administered once or multiple times and the timing adding each cytokine can be controlled properly. Preferably, all the cytokines can be treated once while the cell is cultivated. Preferably, the CD8 T cell is further cultivated for 1 to 4 days even after the cytokines are added.

In the present invention, the simple and effective method for stimulating a CD8 T cell activity that is an important factor determining the efficiency of cell therapy without any toxicity is provided. The resulting CD8 T cell activated by the above-mentioned method can be applied to treat various kinds of cancers, virus infections and immune diseases, as a biological medicine for multiple uses.

In another embodiment of the present invention, a therapeutic composition for preventing or treating infectious diseases of virus that comprises the CD8 T cell activated by the above-mentioned method is provided.

In the other embodiment of the present invention, a method for preventing or treating infectious diseases of virus by administering a therapeutically effective amount of the CD8 T cell activated by the above-mentioned method to a patient.

In the description of the present invention, “prevention” designates all behaviors that inhibit a viral infection or delay an invasion by administering the CD8 T cell activated above to a patient.

In the description of the present invention, “treatment” means all behaviors that improve and advantageously modify a symptom of viral infection by administering the CD8 T cell activated above to a patient.

In the description of the present invention, “patient” denotes human and animals including cow, horse, sheep, pig, goat, camel, antelope, dog and the like that can be improved in infectious diseases of virus by administering the CD8 T cell activated above. The infectious diseases of virus can be prevented or treated efficiently by administering the CD8 T cell activated above in a cytokine mixture comprised of GM-CSF, IFN-γ, TNF-α, lectin, IL-2 and/or IL-4 to a patient.

In the method of the present invention, the infectious disease that can be treated by administering the CD8 T cell activated above is not limited particularly, but preferably an infectious disease caused by a virus pertaining to Orthomyxoviridae family. Preferably, the virus pertaining to Orthomyxoviridae family can be Influenzavirus A, Influenzavirus B, Influenzavirus C, Thogotovirus, Isavirus and the like. More preferably, the infectious disease is an infectious disease caused by Influenzavirus A, Influenzavirus B or Influenzavirus C.

The CD8 T cell can be administered through any pathway, if possible to reach a target tissue. In detail, the cell can be administered parenterally and for example, it can be utilized by intra-peritoneal injection, intravenous, intramuscular, subcutaneous or intra-dermal injection, but it is not limited. The cell composition of the present invention can be administered by using any apparatus possible to move active ingredients to a target cell. The cell composition can be additionally comprised of conventional pharmaceutical carriers suitable for cell therapy such as physiological saline solution.

The CD8 T cell of the present invention should be administered in a therapeutically effective amount. In the description of the present invention, “therapeutically effective amount” designates an amount sufficient to treat diseases in a reasonable ratio of benefit/risk suitable for medical therapy. The dosage to be ingested will vary, depending on factors such as severity of disease, age, sex, time of administration, method of administration, ratio of discharge, period of treatment, other drugs and medical factors already disclosed in this art. It is important to administer a minimal amount effective in a maximal extent without any adverse action, considering all the factors. The dosage may be determined by those skilled in this art. As a general guide, it is expected that adult patient would ingest once about 1 mg to 1,000 mg of the CD8 T cell according to the present invention. The individual patient with a particular body weight and life style may readily determine the proper dosage by starting out with the general dosage level set forth above and adjust the dosage as necessary to alleviate the disease.

Practical and presently preferred embodiments of the present invention are illustrated as shown in the following Examples. However, it will be appreciated that those skilled in the art, on consideration of this disclosure, may make modifications and improvements within the spirit and scope of the present invention.

### EXAMPLE 1

### Preparation of CD8 T Cell Alleviating Influenza Infection

Spleens of mice (BALB/c, SLC Japan) were obtained and sonicated with a cell grinder. The resulting cells suspended in RPMI medium were centrifuged at 1,500 rpm and then, 10 ml of RBC lysing buffer (Sigma, USA) was added to react for 10 minutes at room temperature. After that, the resultant was centrifuged at 1,500 rpm to remove erythrocytes by exchanging 10 ml of RPMI media three times. The mononuclear immune cells obtained above were separated by performing MACS method using anti-CD8 T cell antibodies to obtain CD8 T cells. The resulting CD8 T cells were cultivated for 48 hours with RPMI media containing 10% FBS and cytokines.

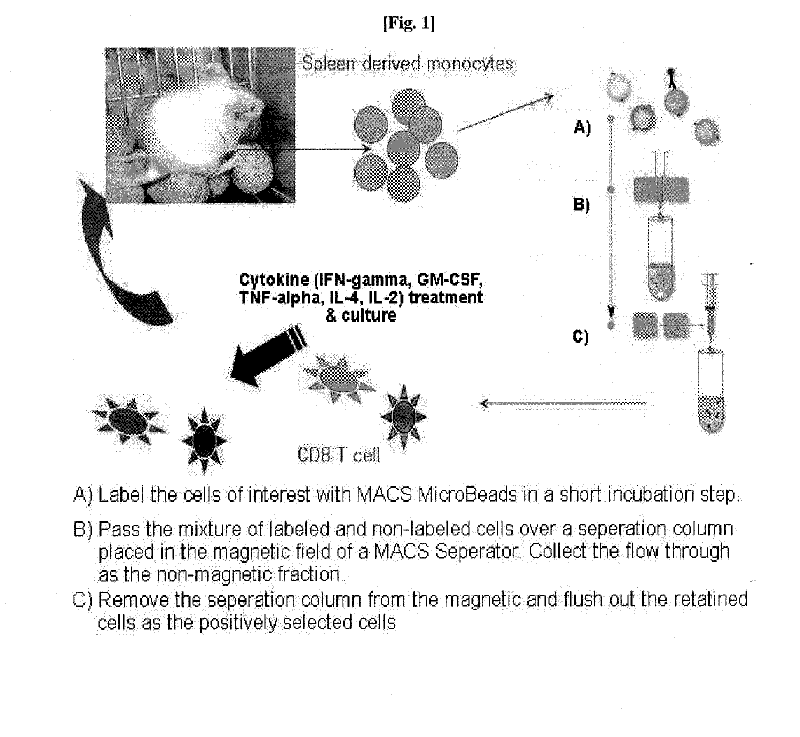

The cytokines added were adjusted in their amounts to GM-CSF 0.1 μg, IFN-γ 1 μg, TNF-α 0.1 μg, lectin 50 μg, IL-2 0.1 μg and IL-4 0.1 μg respectively. The cultured cells were collected and then, suspended with PBS buffer before applied for cell therapeutics. FIG. 1 depicts the process for activating the CD8 T cell after separating it from a subject schematically.

### EXAMPLE 2

### Examination of Efficacy of Cell Therapeutic after Influenza Infection in Mouse Model

In order to induce a viral infection, 1×10⁴ of Influenza A virus (A/PR/8/34, professor Sung Baek Lin of Yonsei University) were bronchially administered in mice. By the same procedure described in the Example 1, CD8 T cells were activated and suspended with PBS buffer to reach 1×10⁶ of cell concentration. Then, the resulting cells were injected intravenously on experimental mice infected by Influenza virus.

The survival ratios of mice were measured and compared in the control group injecting only PBS buffer and the experimental group injecting a macrophage cell line or the CD4 T cell (See Table 1).

TABLE 1

| | control | macrophage | CD4 T cell | CD8 T cell |
|---|---|---|---|---|
| | (survived | (survived | (survived | (survived |
| day | mice/all mice) | mice/all mice) | mice/all mice) | mice/all mice) |
| 2 | 10/10 | 10/10 | 10/10 | 10/10 |
| 4 | 10/10 | 10/10 | 10/10 | 10/10 |
| 8 | 6/10 | 10/10 | 10/10 | 10/10 |
| 10 | 0/10 | 6/10 | 8/10 | 8/10 |
| 12 | 0/10 | 2/10 | 2/10 | 6/10 |
| 14 | 0/10 | 0/10 | 0/10 | 6/10 |
| 16 | 0/10 | 0/10 | 0/10 | 6/10 |

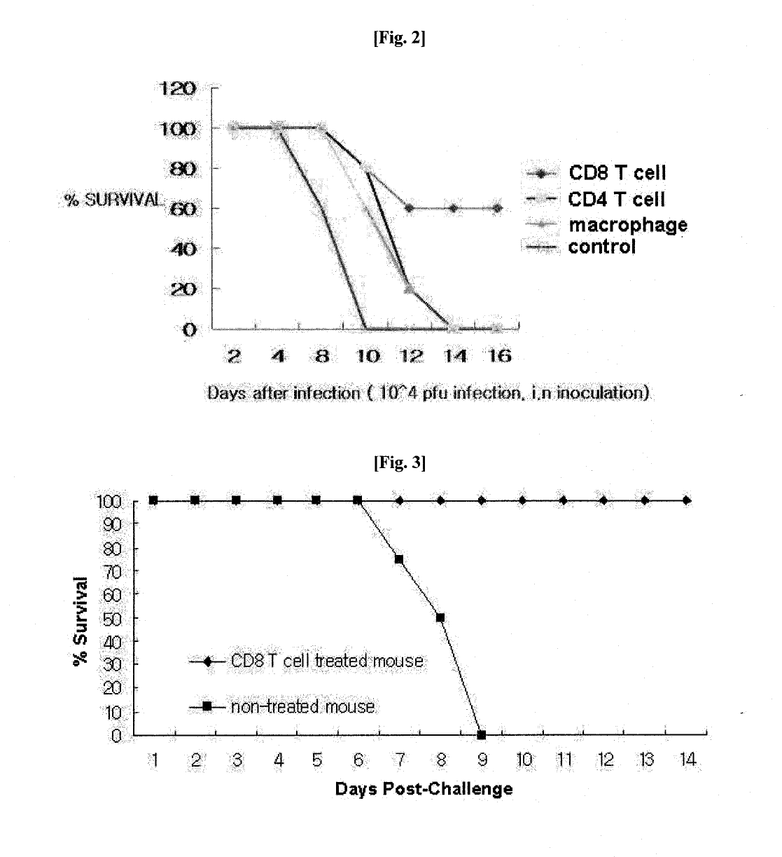

FIG. 2 depicts the comparison of cell efficacies on mice infected by Influenza virus. As a result, it is observed that the CD8 T cell activated above increases a survival ratio of the mice infected by Influenza virus after it is administered.

### EXAMPLE 3

### Examination of Vaccine Efficacy of Cell Therapeutic on Infectious Disease of Influenza in Mouse Model

Experimental mice were infected by Influenza virus and then, treated with the CD8 T cells activated by the same procedure described in the Example 1. 6 mice of the survived group above were administered again with 1×10⁴ of Influenza virus. In contrast, 6 mice of the control group that has never been infected by Influenza virus and injected with only PBS buffer were administered with Influenza virus as described above. Survival ratios were compared in the 2 groups to evaluate an efficacy of the vaccine after administering the CD8 T cells.

FIG. 3 depicts the test of vaccine efficacy on Influenza infection in mice survived after administering the activated CD8 T cell. As a consequence, it is confirmed that the experimental mice survived above is prevented from the re-infection of Influenza virus completely by injecting the CD8 T cell of the present invention.

### EXAMPLE 4

### Examination of Antigen-Antibody Reaction Against Influenza Virus

In order to investigate the vaccine efficacy of a cell therapeutic, the experimental mice were treated with the cells and if survived, operated to collect blood from eyeballs. The blood of mice was centrifuged at 3,000 rpm in order to obtain sera. The resulting sera were examined to measure the degree of antibody reaction specific for an antigen of whole Influenza A virus by using ELISA method.

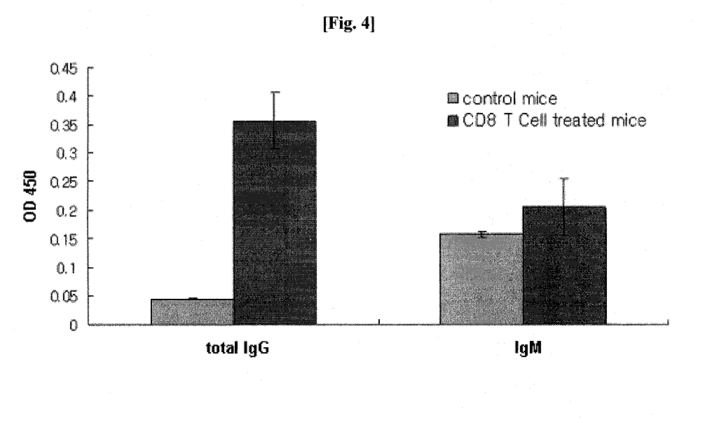

FIG. 4 depicts the antigen-antibody reaction against Influenza virus in sera collected from mice survived after administering the activated CD8 T cell. As illustrated in FIG. 4, it is observed that the mice treated with the CD8 T cells indicates the antibody reaction (total IgG) approximately 7-fold sensitive to Influenza virus, compared to the normal mice without any treatment. Further, it is examined that the mice treated with the CD8 T cells have a higher IgM value, an immunity index of viscous membrane, compared to the normal mice. Therefore, it is confirmed that the experimental mice treated with the cell therapeutic may stimulate an antibody reaction specific for Influenza virus.

As illustrated and confirmed above, the CD8 T cell activated by the method of the present invention will treat and prevent infectious diseases of virus including Influenza virus, etc effectively.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method for in vitro activation of a CD8 T cell, comprising:
separating CD8 T cells from a biological specimen of subject; and
cultivating the CD8 T cells with a culture medium containing cytokines including GM-CSF, IFN-γ, TNF-α, lectin, IL-2 and IL-4.

2. The method according to claim 1, wherein the biological specimen is one selected from a group consisting of blood, plasma, lymph node, spleen, thymus and bone marrow.

3. The method according to claim 1, wherein the concentration of cytokines are adjusted in the ranges of 0.05 to 0.2 μg/ml of GM-CSF, 0.5 to 2 μg/ml of IFN-γ, 0.05 to 0.2 μg/ml of TNF-α, 40 to 60 μg/ml of lectin, 0.05 to 0.2 μg/ml of IL-2 and 0.05 to 0.2 μg/ml of IL-4.

4. The method according to claim 1, wherein the cultivating includes cultivating the CD8 T cells for 1 to 4 days.

5. A therapeutic composition for preventing or treating infectious viral diseases, comprising the CD8 T cells activated by the method of claim 1.

6. The therapeutic composition according to claim 5, wherein the virus is a virus pertaining to Orthomyxoviridae family.

7. The therapeutic composition according to claim 6, wherein the virus is Influenzavirus A, Influenzavirus B or Influenzavirus C.
